(19) United States
(12) Patent Application Publication
(10) Pub. No.: US 2026/0111801 A1
(43) Pub. Date: Apr. 23, 2026

(54) Assessment of Correlatable Data

(71) Applicant: Mustafa Behan, Berlin (DE)

(72) Inventor: Mustafa Behan, Berlin (DE)

(21) Appl. No.: 19/424,200

(22) Filed: Dec. 18, 2025

Related U.S. Application Data
(63) Continuation-in-part of application No. 18/459,030, filed on Aug. 30, 2023, now Pat. No. 12,530,626.

(51) Int. Cl. G06N 20/00; G16H 50/20

(52) U.S. Cl. CPC G06N 20/00; G16H 50/20

(57) ABSTRACT

Disclosed is a biomarker digitized content assessment approach using online and centralized features to train a machine learning assessment model implemented to assess the correlation of certain biomarker digitized content for improving diagnostic outcomes, assessing risk, and monitoring treatment status. The machine learning assessment model generating identification of biomarker digitized content based on a relational technology that identifies, compares, and assesses connections between digital content elements (such as time patterns, sentence structure, proximity, characterization similarities, etc.) to build an intelligent assessment tool that identifies singularities and patterns within sets of digitized content elements to identify relational sources of digital content. The model creating an assessment of the relationship of digital content by screening digital content against peer data records, the screening multiplied according to a plurality of iteration steps. Singularities corresponding to linkages may be weighted according to various known biomarker characteristics.

This continuation-in-part application, under 35 U.S.C § 120, claims the benefit of U.S. patent application Ser. No. 18/459,030 filed Aug. 28, 2023.

### TECHNICAL FIELD

The present disclosure relates to training and using machine learning assessment and decision models that analyze digitized data from various origin points to identify data connections and, more particularly, an assessment and decision model that identifies singularities through various operations of the digitized data process in order to identify patterns in the healthcare environment to improve diagnostic and treatment methodologies.

### BACKGROUND

Patient healthcare data is often examined in isolation. Medical tests and their results (healthcare data) generally look at a single aspect of overall health. The analysis of these results happens sequentially and independently from one another. Data is not normally reviewed in connection to each other within the context of the total patient history. Further, data, whether a single test result or in conjunction with other healthcare data, is not analyzed in conjunction with peer group analysis of comparable patients with known disease trajectories.

The present invention advances the field of patient diagnosis and treatment by codification of data sets and subsequent mathematical operations on the codified data that includes target patient data and data points of other patients. The present invention further identifies implicit connections between datasets on non-linear logical spaces. Through codification and connection identification, the present invention further identifies and analyzes data for singularities and indications for elevated risks, progression of diseases, and further testing as compared to known data points, The present invention thereby improves the accuracy and speed of diagnosis, treatment effectiveness, and risk analysis.

### SUMMARY

Various embodiments of the disclosure relate to a machine learning method which may be implemented by a computing system of a data integrity systems operator. The method may comprise generating biomarkers to a model of known peer data points, the relationships between the known peer data points and the biomarker to assess the potential for elevated risks, disease progression, and indication of further testing. The method may comprise sending the digital content to a machine learning analytical model to generate an authenticity assessment of certain digital content. The machine learning assessment model may have been trained by applying machine learning to analytical models and digital content to synthesize machine learning to understand large sets of digital content.

Various embodiments of the disclosure relate to a machine learning computing system comprising a processor and a memory comprising instructions executable by the processor. The instructions may comprise generating markers for various data by processing the digital content in a model of known peer data points and identifying the relationships between them and the data in question.

The various data field content may come from disparate data groups having data points, that when connected, may produce a data output of related data points which reveals insights to the patients'medical status, confirms or disproves a diagnosis—and indicate for further testing. The machine learning platform may comprise sending the digital content to a machine learning assessment model to generate an assessment of correlated digital content. The machine learning assessment model may have been trained by applying machine learning to a set of data points with corresponding known peer data points.

Various embodiments of the disclosure relate to a machine learning method which may be implemented by a computing system of a data integrity systems operator. The method may comprise generating data marker indicators by identifying singularities to develop known peer data points and apply them to identify and assess the markers in question. The method may comprise sending the markers to a machine learning assessment model to generate an assessment. The machine learning assessment model may have been trained by applying machine learning to network features and non-network features.

In various example embodiments, the network model may comprise a biomarker. Throughout, biomarker can refer to, without limitation, molecular, physiologic, histologic, or radiographic sources, and can be selected from one or more of blood, urine, tissue, sputum, x-ray, CT, MRI, Ultrasound, fecal matter or other sources like sleep data, blood pressure, heart rate or motion videos of a patient. The term biomarker, whether identified in the singular or in the plural refers to one or more biomarkers. The machine learning platform may define at least one singularity in one or more biomarkers according to at least one of a distance between a biomarker and time lags between responses setting a fingerprint and identifying patterns between biomarker and known peer data points. The machine learning platform translates each biomarker into a metrical value to identify patterns and similarities within a set of biomarkers. These patterns are translated into an assessment of similarities to understand common sources of impact on biomarkers—which may then be used to increase the accuracy of diagnosis, treatment status or risk assessment. These common sources could then be used to identify an underlying reason for a health condition, such as a tumor, virus, or other medical singularities like increased risks for diseases.

In various example embodiments, the network model may comprise histology testing results. The machine learning platform translates each result into a metrical value to identify patterns and similarities within a set of histologic test results. These patterns are translated into an assessment of similarities to understand common patterns within peer data points.

In various example embodiments, the model may comprise molecular testing results. The machine learning platform may translate each result into a metrical value to identify patterns and similarities within a set of peer data points. These patterns are translated into an assessment of similarities to understand common patterns within peer data points.

In various example embodiments, the network model may comprise physiologic testing results. The machine learning platform may translate each result into a metrical value to identify patterns and similarities within a set of peer data points. These patterns may then be translated into an assessment of similarities to understand common patterns within peer data points.

In various example embodiments, the network model may comprise imaging testing results. The machine learning platform may translate each result into a metrical value to identify patterns and similarities within a set of peer data points. These patterns may then be translated into an assessment of similarities to understand common patterns within peer data points.

In various example embodiments, the model may comprise distance operators. Known data points may comprise distance points for determining distance and/or time between biomarkers and known peer data points. The machine learning platform may be configured to generate biomarker data points by creating data points for combinations of distance operators and comparable known data points.

In various example embodiments, the model may comprise a network corresponding to linkages between known data points. The machine learning platform may translate each linkage into a metrical value to identify patterns and similarities within a set of linkages. These patterns may be translated into an assessment of similarities to understand common sources of information-which may then be used to identify connections between various biomarkers, as the case may be, for increasing diagnostic accuracy or treatment status.

Various embodiments of the disclosure relate to a machine learning method implemented by a computing system of a data integrity system operator. The method may train one or more assessment models for assessing biomarker data to determine its validity, thereby increasing the effective biomarker accuracy for making a diagnosis or when determining treatment status. The method may comprise generating a model of known peer data points and correlating relationships between biomarker data points and the known peer data points. The network model may comprise at least one data unit (e.g., biomarkers from various modalities, such as, urine, feces, blood, radiographic, tissue, etc.).

The method may comprise generating biomarker data points by applying known peer data points to the model. The method may comprise applying machine learning to the known biomarker data points to train a machine learning assessment model configured to generate assessments of biomarker data points to increase the accuracy of diagnosis or treatment status.

Various embodiments of the disclosure relate to a machine learning method implemented by a computing system for the analysis and assessment of biomarker data points, wherein biomarker data points are received by a computing system and verified. A machine learning assessment model using known peer data points as training data is then engaged, the machine learning assessment model trained to generate identification of singularities in biomarkers. The machine learning assessment model, using the known data points as input to generate an assessment of biomarkers to determine whether it is noticeable, the machine learning assessment model comprising a density-based clustering technique that is a function of a density operator. The machine learning assessment model defining each singularity in the network according to at least one of an amount, a frequency, or an incidence of data points between the received biomarkers and known peer data points. The model comprises distance operators, and wherein generating the peer data records comprises generating, by the computing system, features for combinations of distance operators and distance vectors compatible with corresponding distance operators.

Various embodiments of the disclosure including a machine learning computing system for codification, assessment, identification and verification of biomarker data points, the computing system comprising a processor and a memory comprising instructions executable by the processor, the instructions comprising a machine learning platform configured to train a machine learning assessment model using known peer data points, in whole or in part, as training data, the machine learning assessment model trained to generate assessments of biomarker data points to increase the accuracy of diagnosis or treatment status. Execution by the machine learning assessment model using the known peer data points as input to generate an assessment of biomarkers. The model screening biomarker data points at least once for singularities, wherein the machine learning platform defines each singularity according to at least one of a frequency or an incidence of known peer data.

The model including a network corresponding to linkages between known peer data records, including data points, wherein the machine learning platform defines each singularity in the network as indicating a linkage between corresponding tuples of known peer data points and digital data, each singularity weighted according to a characteristic of the corresponding linkage.

These and other features are explained in more detail in and will become apparent from the following detailed description and the accompanying drawings.

### BRIEF DESCRIPTION OF THE DRAWINGS

FIG. 1 is a flow diagram of a general overview of the system.

FIG. 2 is a block diagram of a data integrity system operator computing system that implements a machine learning platform and that communicates with third-party platforms.

FIG. 3 is a process flow diagram for a machine learning approach to detecting singularities in biomarkers.

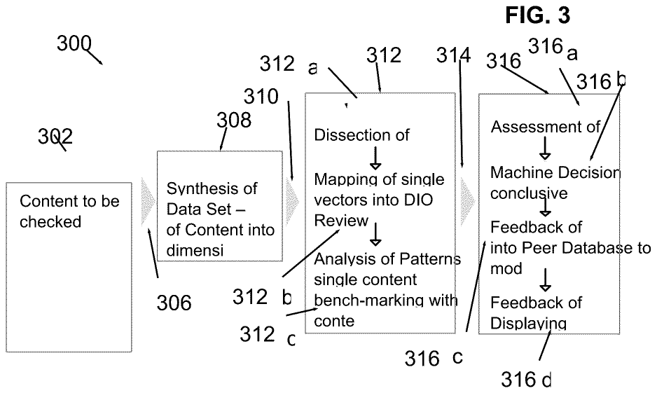

FIG. 4 is a decision tree diagram illustrating the steps of biomarker analysis.

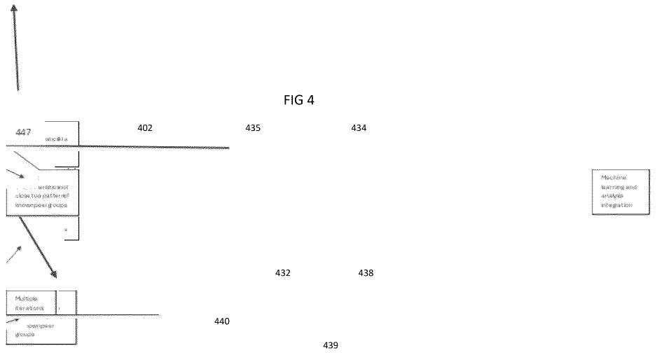

### DETAILED DESCRIPTION

Healthcare data is most often in the form of biomarkers. Biomarkers are observable indicators of a biological state or condition. Biomarkers can provide valuable information about a person's health status, including the presence or absence of certain diseases. They can be molecular, physiologic, histologic, or radiographic, and their measurements are used for diagnosis, predicting risk, and monitoring disease progression or treatment effectiveness. Common examples, without limitation, include blood pressure, genetics, proteins levels, radiological tests, or other substances in the blood and body fluids.

Viewed from a digitized data standpoint, the analysis of biomarkers can hint to a “manipulative entity” that creates singularities in the data indicate that there is an abnormality that suggests disease progression and the need for further testing.

In the case of health care and checking of medical data, a specific disease or combination of diseases is mathematically viewed also as the manipulative entity that will create singularities that can be detected.

The medical data, such as results of complete blood counts, multi-dimensional health data (e.g. AST/ALT in combination with BMI, blood sugar data, thyroid test, and blood oxygen, lactic acid) is checked against a timeline of datapoints of the same patient as well as datapoints from comparable patients to identify singularities.

These data points are structured and translated into metrical values to identify singularities and patterns in repetitions or singularities in mismatches. A disease and the boundary conditions of the patient (such as lifestyle factors, pre-dispositions, environmental facts) is then mathematically viewed as an entity that (1) creates discontinuities and (2) repeats itself.

As described herein, these are recognized as singularities. The significance of these singularities are analyzed to assess whether the patient data matches already known patterns of singularities. These singularities are then mapped to indicators for specific disease progression trajectories. This is used to predict disease progression, plan for interventions, and define a schedule for necessary scanning and medical check-ups.

In the various embodiments presented herein, machine learning approaches, which may involve supervised and/or unsupervised learning, may be used for training and implementing assessment machine learning models for detecting, analyzing and correlating one or more biomarkers against known data points for increasing the accuracy of disease diagnosis or treatment status.

Biomarkers measure characteristics that indicate a health condition or response to treatment. Accordingly, throughout, the term digitized or digital data includes at least one biomarker from any source or by any means, without limitation, including genetic testing, blood, urine, feces, sputum, radiographic (including without limitation, x-ray, ultrasound, CT, MRI, etc.), tissue, etc.

A representation of biomarker data and its relationships to a known peer group of data points can be determined and used to translate identified data points into bench-marking peer data points. Machine learning models may be applied to analyze connections and understand relations between different data points and thereby more effectively and efficiently indicate connections between biomarker data in order to increase diagnostic accuracy, assess risk or measure treatment status. As those skilled in the arts will understand, this benefit can assist to more specifically create treatment protocols, while at the same time reduce the need for more invasive procedures, such as biopsies, and also reduce patient discomfort and costs.

A machine learning platform trains and uses models to exploit the fact that certain biomarker data points are specific to a particular disease state. Each singularity of the biomarker data may be defined as containing a linkage between known biomarker data points which, in turn, may be weighted according to one or more characteristics of the known peer group data points. A singularity indicating a linkage between corresponding tuples of known peer data points and the biomarker data in question, wherein the computing system defines each singularity as denoting a linkage between corresponding tuples, pairs are sets of two, n-tuples are set of n elements of biomarker data, and each singularity weighted according to a characteristic of the corresponding biomarker data. The method may comprise generating data points by applying known peer data points to the model. The method may comprise applying machine learning to the known peer group data points to train a machine learning assessment model configured to generate assessments of biomarker digital content, and which may be described by distance operators.

In various example embodiments, the method may comprise comparing biomarker data points corresponding to known peer group data points to generate an assessment of the biomarker data in question.

The model may comprise distance operators that operate on a curved-space. Unlike orthogonal matrices which generate results based only on angled vectors of 90 degrees thus creating a matrix, the herein developed and applied distance operators enable a broader scope of activity by enabling a curved inclusion area, thereby offering the opportunity for additional data point recognition. The use of these distance operators further enhances the ability to interpret digital data points using multiple peer data points and known biomarker data points. The known biomarker data points may comprise codifying from combinations of distance operators and corresponding known peer group data points to identify similarities in certain biomarker data.

The machine learning platform may employ a variety of machine learning techniques in classification and pattern recognition of digital data points to determine “unimpacted” (as opposed to “impacted”) patterns in data and to identify unusual outlier data points.

Impacted biomarker data is often not discoverable through analyzing the individual biomarker data in isolation, but through data point relationships and linkages with peer group data points. Whether the peer group data points are related or not is immaterial; the pattern/distance between data points provides the necessary information to make an assessment. Certain activities may disguise related biomarker digital content unless the activities are placed in the context of known peer group data points with some identifiable association which may, in isolation, seem normal. The disclosed approach thus enables the discovery and promotion of connected biomarker data that could not otherwise be discovered through conventional means. Further, the disclosed approach reveals relevant connections within biomarker data not feasibly discoverable by human users. Training an assessment model using a combination of network-based and other features as disclosed herein places the biomarker data in context rather than being considered in isolation, thereby yielding accurate assessments, thereby improving diagnostic, risk assessment, and treatment status.

Example embodiments of the machine learning model described herein improve computer-related technology by performing functions that cannot be carried out by conventional computing systems. Furthermore, the embodiments described herein cannot be carried out by humans. The machine learning model may proactively compare biomarker data corresponding to known peer group data points and known biomarker data points to identify singularities that would otherwise go undetected. Conventional systems may include databases and definitions that are static and cannot be configured to acquire, store, and update the multitude of information required from identifying peer group data points and known biomarker data points.

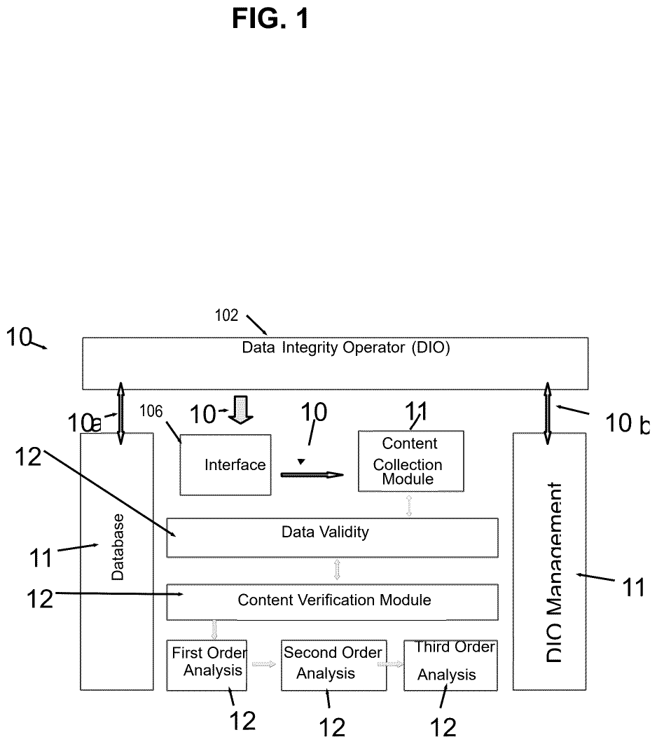

Referring to FIG. 1, a flow diagram illustrating the basic movement of digital content through the inventive system 100 is shown, according to potential embodiments.

The system 100 includes a platform computing system 102, here a data integrity operator (DIO). A DIO may be defined as a centralized server that houses peer group data points in a Database 116. The platform computing system 102 may be that of an online healthcare facility that publishes test data (either through a patient portal or generally with unidentifiable patient data). For example, many clinics and hospitals place patient data into online portals. Other organizations collect non-identifiable healthcare data from individuals. All such data generally contains results that can be identified as biomarkers. These biomarkers, without reference to any particular individual, can provide data points that may be useful in correlating with other known data points. When used in aggregate, these known data points may be correlated with other known data points to increase the accuracy of testing.

This can, in turn, reduce false-positive and false-negative results caused by overly sensitive tests, issues with sample collection, or technical artifacts that can lead to unnecessary anxiety, follow-up procedures, and costs.

Also important to accurate diagnosis and risk assessment is to minimize false positives, which require proper test validation, which involve considering factors like sensitivity and specificity, using independent datasets, and being cautious when interpreting results in the context of a patient's overall health. Current common techniques of validation involve two main areas: analytical validation, which ensures the test accurately measures the biomarker, and clinical validation, which confirms the biomarker's meaningful association with a health outcome. Key techniques include using platforms like ELISA and qPCR for analytical validation, applying statistical methods and resampling techniques for internal validation, and conducting retrospective and prospective studies to evaluate clinical performance across diverse patient populations.

Statistical validation is commonly used to confirm a biomarker's predictive power and its correlation with the clinical endpoint. Techniques include using bootstrapping or cross-validation on development data (internal validation) and then confirming results with completely independent datasets (external validation).

The DIO 102 further comprises memory hosting a database 116 for housing the data points and a DIO Management Module 118 for operating the system. Database 116 and Management Module 118 communicate with DIO 102 at communication points 102a and 102b, respectively.

The component parts of the system 100 may be integrated or operatively coupled to one another directly or over a network that permits the exchange of data and the like. System 100 may include one or more processors, memories, network interfaces, and user interfaces. The memory may also store data points in database 116. Interface 106 allows system 100 to communicate 104 with DIO 102, when operatively coupled, by sending and receiving transmissions via one or more communications protocols.

Once a platform computing system 102 communicates 104 at Interface 106, the digital content submission and analysis process is initiated by the computing system. Available biomarkers are checked for scope (what kind of data is available) and checked for validity (is the data complete, sound and within pre-defined intervals). The data validity check 120 is an order check of the biomarker data itself, without comparison to any other content or data point. Validity is determined by analyzing basic features of the biomarker data such as completeness of test result, appropriateness of testing for target result, etc. A biomarker may fall out or move on further in the analysis process. In either event, verification check 122 is initiated. For biomarker data that are disqualified in the data validity check, data points are collected and stored in data base 116. Disqualification results in the biomarker data not being used. If there is an impacted biomarker data array, this will be stored in the database to train the system how other impacted biomarkers look like. The data is then combined to create a coded biomarker array for each data subject that needs to be assessed. As a coded biomarker array, the mapping of biomarkers into a multi-dimensional, non-orthogonal and curved space to an n-tuple of coded data can be undertaken. The coding of the data as well as the metrics of the space are determined by operators based on the learning data input into the system. This is refined with any new array that is added to the known data points.

Features such as biomarker limit norms (ranges, etc.), are recorded and analyzed in combination with and against one another to locate patterns in digital content that identify biomarker data points

Once the coded biomarker array is completed, a first order analysis 124 is performed using distance operators of the first order by the model wherein data points for each biomarker are codified and then using the different known peer group data points compared to other known peer group data points. A determination is made by measuring the distance of data points against known peer group data points. Through multiple iterations, at least two but that may number up to 70 or more, further determination can be made.

Further analysis is initiated by the model using distance operators of the second order 126 wherein measurement may identify patterns between groups of biomarker arrays. Distance operators of the second order calculate the distance between one single biomarker array and a set of arrays. These distance operators identify both similarities of single biomarkers with a group or singularities of biomarkers. The second order analysis may look at any forms of patterns. Such analysis is not easy to identify in one single piece of content, but by looking at patterns in the peer group. Again, the second order analysis is performed through multiple iterations numbering up to 70 or more.

A final analysis is performed using distance operators of a third order wherein the outcomes of the first order distance operator and second order distance operator analysis are analyzed for significance to calculate a confidence and severance level for the identified impact. Distance operators of the third order calculate the significance of similarities or singularities between biomarkers to understand whether these biomarkers indicate a noticeable observation or a conclusion for a medical indication. If the significance determined by the distance operator of the third kind is above a defined threshold, we have identified an impact.

An impact creates a deviation of the biomarker array from expectable distances to defined biomarker arrays or distances to a defined biomarker set with a level of significance. The impact would then indicate that the examined patient has a health driver that alters the expected tuple. By this method, the healthcare provider will identify an area of concern or further investigation. The levels of significance are calculated through learning data (known data points) and any new biomarker added to the database.

The machine learning model of the present invention is premised on the hypothesis that:

Step 1: Data pre-processing. Check the scope of the available data (what kind of data is available). Check all available data for integrity (are the data complete, sound, and within pre-defined intervals). In other words, does it make sense to process this data or should we go back to the data source.

Step 2: Building the coded biomarker array. All available patient data are coded along a curved, multi-dimensional, (non-orthogonal) space into an n-tuple of data.

Step 3: First order check using distance operators of the first order.

Understand whether the coded biomarker array is close to a known benchmark in the database where an impact was previously already identified.

Step 4: Second order check using distance operators of the second order. Understand whether the coded biomarker is close to a set of biomarker arrays that suggest an impact (with less certainty).

Step 5: Third order check using distance operators of the third order.

Understand the significance of the calculations done in step 3 and step 4 to calculate a confidence and severance level for the identified impact.

Step 6: Deriving an analysis for the medical professional—for example for further testing and a forecast of the patient trajectory. The results of more conclusive tests can be fed back to the system and stored in the database for learning.

Step 7: Using the result of the calculations and potential further information to improve the algorithm by adjusting the metrics of the data space.

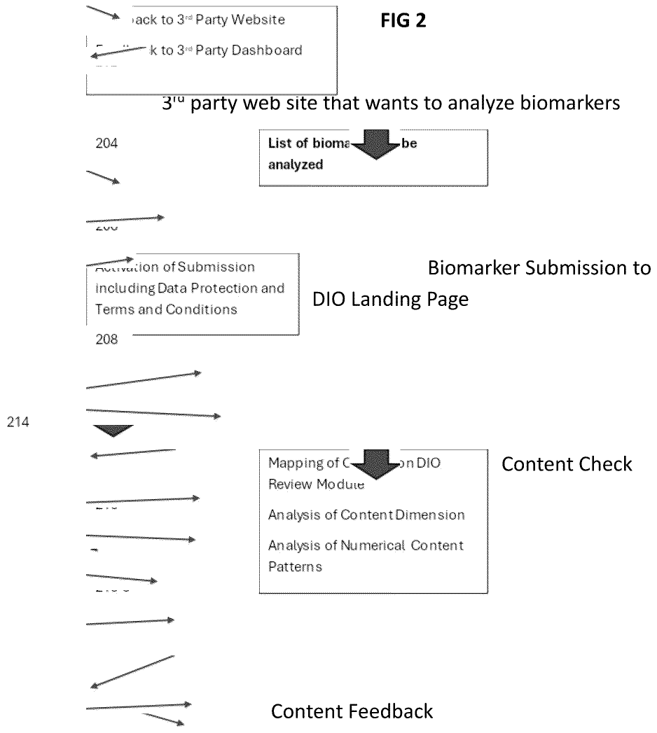

Turning to FIG. 2, is a depiction of a system 200 flow example of a DIO working with a third-party site that hosts biomarker data. For example, in addition to hospitals and clinics, there are third party electronic medical record services. Third party 202 contacts 206 DIO website through its landing page 208. Third party 202 interfaces DIO through a UI wherein it signs up with DIO and submits biomarker data 210a. Once a biomarker data submission is validated 210B, third party 202 sends DIO digital content for analysis 214 using the machine learning model 216. Mapping of third-party biomarker data 216a is performed by the model using DIO's data point data base as implemented by a computing system employing the machine learning model 216b, first order analysis 216d and second order analysis 216d, as discussed above, is performed by the computing system (see discussion above, FIG. 1). A determination is made as to whether the digital content is connected 216d. The determination is then transmitted 218 to the third party 202 as feedback 220, 222 through third party's website and then to DIO dedicated dashboard 222b. Third party retrieves the feedback from its dashboard and makes an internal decision or use the proposed decision developed by the computing system whether to include the biomarker data.

Thereafter, machine learning system 300 provides feedback of the assessment to the peer group database to train the model 316c while also providing feedback to the requesting website 316d.

If biomarker data characteristics (data points) are similar to peer group known data points 444 content characteristics will be stored within the database 116 (FIG. 11) for machine learning model learning purposes and later analysis integration 447. In the case that digital content data points are different from unremarkable known peer group data points 432, the digital content data points are further checked for content characteristics within known peer groups 434, i.e., second order analysis. If digital content characteristics are not part of a pattern, content characteristics will be stored within the database 116 (FIG. 11) for machine learning model learning purposes and later analysis integration. In the cases where digital content data point characteristics are close to a pattern of known unremarkable data points 440 At machine learning process steps 432, 434, multiple iterations are undertaken. Typically, there are between 70 and 200 data points per peer digital content. However, with biomarkers, particularly if they include image creating tests and timelines, this number could be easily greater than 100,000. Each new submission adds to that count and each new submission is reviewed against all known group peer biomarkers. Thus, each new digital content submission may be analyzed at each of these two steps between 70 and 200 times multiplied by the number of biomarkers in the peer group. Though a range is provided, it is not meant to be limiting as the number of iterations is dependent on the number of biomarkers and known peer groups.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principles of the disclosure and its practical application to enable one skilled in the art to utilize the various embodiments and with various modifications.

Example computing systems and devices may include one or more processing units each with one or more processors, one or more memory units each with one or more memory devices, and one or more system buses that couple various components including memory units to processing units. Each memory device may include non-transient volatile storage media, non-volatile storage media, non-transitory storage media (e.g., one or more volatile and/or non-volatile memories), etc. In this regard, machine-executable instructions comprise, for example, instructions and data which cause a general-purpose computer, special purpose computer, or special purpose processing machines to perform a certain function or group of functions. Each respective memory device may be operable to maintain or otherwise store information relating to the operations performed by one or more associated modules, units, and/or engines, including processor instructions and related data (e.g., database components, object code components, script components, etc.), in accordance with the example embodiments described herein.

It should be noted that although the drawings herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims.

Such variations will depend on the machine-readable media and hardware systems chosen and on designer choice. It is understood that all such variations are within the scope of the disclosure. Likewise, software and web implementations of the present disclosure may be accomplished with standard programming techniques with rule-based logic and other logic to accomplish the various database searching steps, correlation steps, comparison steps and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principles of the disclosure and its practical application to enable one skilled in the art to utilize the various embodiments and with various modifications as are suited to the particular use contemplated. Other substitutions, modifications, changes and omissions may be made in the design, operating conditions and arrangement of the embodiments without departing from the scope of the present disclosure as expressed in the appended claims.

What is claimed:

1. A computer-implemented machine learning method for the connection of biomarker digitized content in an electronic system, the method comprising:
receiving, by a computing system comprising one or more processors and a memory storing executable instructions, biomarker digitized content submitted to a network-accessible content platform;
verifying the digital content by:
(i) capturing a plurality of relevant dimensions of the digitized content according to a predefined protocol for digital content capture;
(ii) converting the captured digitized content into structured data;
(iii) generating an initial integrity assessment of the structured data;
(iv) translating the structured data into a plurality of characteristic parameters;
(v) performing an intrinsic singularity analysis of the digitized content based on the characteristic parameters;
(vi) performing a metrical analysis using distance operators of distances between the digitized content and previously stored digitized content in a database to simulate a network of content after adding the digitized content to the network by checking the distances between all datapoints in the network that would be established after adding the new datapoint to identify singularities for application to known data points;
(vii) applying one or more distance operators to the computed distances;
(viii) generating a singularity decision for the digital content; and
(ix) updating a machine learning system with the singularity decision for use in subsequent analyses of future digitized content;
training, by the computing system, a machine learning assessment model using known peer group data points as training data, the machine learning assessment model configured to perform correlation analysis of biomarker digitized content;
generating, by the computing system, based on a plurality of data records accessed from an electronic database, a biomarker-detection model comprising a set of patterns and relationships among the known peer group data points that are indicative of biomarker digitized content;
retrieving, by the computing system, from the electronic database, a plurality of the known peer group data points;
generating, by the computing system, an assessment analysis of the digital content by screening the digital content against the retrieved peer group data points, the screening being performed over a plurality of iteration steps;
executing, by the computing system, the machine learning assessment model using the known peer group data points as input to produce an assessment of whether the digital content is relevant to other biomarker known peer data points, wherein the machine learning assessment model comprises a density-based clustering technique defined by a density parameter; and
generating, by the computing system, an indicator marking the biomarker digitized content as relevant in response to the assessment indicating correlated biomarker content,
wherein the indicator is automatically applied by a content relational system to promote the correlation of digitized content, thereby improving the accuracy and efficiency of automated digitized content correlation on the network to increase the relational aspects of biomarkers.

2. The method of claim 1, wherein the predefined protocol for digital content capture comprises extracting at least one of: textual features, metadata attributes, embedded file signatures, pixel intensity histograms, or compression artifacts.

3. The method of claim 1, wherein the one or more distance operators comprise normalization functions that scale computed distances into a probabilistic correlation score.

4. The method of claim 3, wherein the density parameter of the density-based clustering technique is dynamically adjusted based on at least one of: average distance between cluster points, variance in content similarity scores, or number of connected components in the network.

5. The method of claim 1, wherein training the machine learning assessment model comprises performing distributed training across a plurality of GPU-enabled computing nodes.

6. The method of claim 1, wherein generating the indicator marking the biomarker digitized content as relationally relevant further comprises embedding the indicator as a machine-readable tag in the metadata of the content.

7. The method of claim 1 wherein biomarker digitized content is selected from the group comprising molecular, physiological, histologic, or radiographic results.

8. A computing system for assessment of biomarker digital content in an electronic system through the identification of singularities, the system comprising:
one or more processors; and
a memory storing executable instructions that, when executed by one or more processors, verifies the digital content by:
(i) capturing a plurality of relevant dimensions of the digital content according to a predefined protocol for digital capture;
(ii) converting the captured digital content into structured data;
(iii) generating an initial correlation assessment of the structured data;
(iv) translating the structured data into a plurality of characteristic parameters to create coded biomarker arrays;
(vi) performing a metrical analysis using one or more distance operators of distances between the biomarker array and previously stored biomarker arrays in a database to simulate a network of content after adding the biomarker array to the network by checking the distances between all datapoints in the network that would be established after adding the new datapoint to identify and assess biomarker array; and,
(vii) updating a machine learning system with the correlation decision for use in subsequent analyses of future digital content;
training, by the computing system, a machine learning assessment model using known peer group data points as training data, the machine learning assessment model configured to perform identification of biomarker array;
generating, by the computing system, based on a plurality of data records accessed from an electronic database, a biomarker relation model comprising a set of patterns and relationships among the known peer group data points that are indicative of relational biomarker array;
retrieving, by the computing system, from the electronic database, a plurality of the known peer group data points;
generating, by the computing system, an assessment analysis of the digital content by screening the digital content against the retrieved peer group data points, the screening being performed over a plurality of iteration steps;
executing, by the computing system, the machine learning assessment model using the known peer group data points as input to produce an assessment of whether the biomarker digital content is relational to known data points for a particular disease state, wherein the machine learning assessment model comprises a density-based clustering technique defined by a density parameter; and
generating, by the computing system, an indicator marking the digital content as relational in response to the assessment indicating relational content,
wherein the indicator is automatically applied by the content publishing system to promote relations of relational digital content, thereby improving accuracy and efficiency of automated content moderation on the network.

9. The system of claim 8, wherein the metrical analysis using one or more distance operators of distances includes the further steps of a first order distance operator for determining whether the coded biomarker array is close to a known benchmark in the database where an impact was previously already identified;
a second order distance operator check to determine whether the coded biomarker is close to a set of biomarker arrays that suggest an impact; and,
a third order distance operator check to determine the significance of the calculations done in in the first order distance operator check and
the second order distance operator check to calculate a confidence and severance level for an identified impact.

10. The system of claim 8, wherein at least one singularity is defined according to at least one of a distance between biomarker array and distances to identify patterns between the biomarker array and known peer biomarker arrays.

11. The system of claim 8, wherein at least one singularity in the network according to at least one of an amount, a frequency, or an incidence of data points between the received biomarker array and known peer biomarker arrays.

12. The system of claim 8, wherein at least one singularity indicating a linkage between corresponding tuples of known peer biomarker arrays and biomarker array.

13. The system of claim 9, wherein the density parameter of the density-based clustering technique is dynamically adjusted based on at least one of: average distance between cluster points, variance in content similarity scores, or number of connected components in the network.

14. The method of claim 8 wherein digital content is selected from the group comprising molecular, physiological, histologic, or radiographic results.

15. A non-transitory computer-readable medium storing instructions that, when executed by one or more processors of a computing system, cause the computing system to correlate biomarker digital content by:
(i) capturing a plurality of relevant dimensions of digital content according to a predefined protocol for digital content capture;
(ii) converting the captured digital content into structured data;
(iii) generating an initial authenticity assessment of the structured data;
(iv) translating the structured data into a plurality of characteristic parameters;
(v) performing an intrinsic authenticity analysis of the digital content based on the characteristic parameters;
(vi) performing a metrical analysis using distance operators of distances between the digital content and previously stored digital content in a database to simulate a network of content after adding the digital content to the network by checking the distances between all datapoints in the network that would be established after adding the new datapoint;
(vii) applying one or more distance operators to the computed distances;
(viii) generating a biomarker relational decision for the digital content; and
(ix) updating a machine learning system with the significance decision for use in subsequent analyses of future digital content;
training, by the computing system, a machine learning assessment model using known peer group data points as training data, the machine learning assessment model configured to perform correlation of biomarker digitized content;
generating, by the computing system, based on a plurality of data records accessed from an electronic database, a biomarker relationship model comprising a set of patterns and relationships among the known peer group data points that are indicative of biomarker digitized content;
retrieving, by the computing system, from the electronic database, a plurality of the known peer group data points;
generating, by the computing system, an assessment analysis of the digitized content by identifying singularities by screening the digitized content against the retrieved peer group data points, the screening being performed over a plurality of iteration steps;
executing, by the computing system, the machine learning assessment model using the known peer group data points as input to produce an assessment of whether the digital content is relationally identifiable with known biomarker data points, wherein the machine learning assessment model comprises a density-based clustering technique defined by a density parameter; and
generating, by the computing system, an indicator marking the digital content as related in response to the assessment indicating related biomarkers,
wherein execution of the instructions further causes the computing system to automatically apply the indicator to promote the correlation of related biomarker digitized content, thereby improving diagnostic accuracy.

16. The non-transitory computer-readable medium of claim 15, wherein the instructions cause the computing system to dynamically adjust a density parameter based on at least one of: average distance between cluster points, variance in content similarity scores, or number of connected components in the network.

17. The method of claim 15, wherein the computing system further comprises a biomarker correlation dashboard configured to display the significance correlation decision and supporting metrics.

18. The method of claim 15, wherein the plurality of iteration steps in screening the digital content comprises performing progressive filtering using increasingly strict similarity thresholds.

19. The system of claim 15, wherein at least one singularity in the network according to at least one of an amount, a frequency, or an incidence of data points between the received digital content and known peer data points.

20. The system of claim 19, wherein at least one singularity indicating a linkage between corresponding tuples of known peer digital content data points and digital content.
